(19) United States
(12) Patent Application Publication
(10) Pub. No.: US 2004/0053967 A1
(43) Pub. Date: Mar. 18, 2004

(54) 3-(3-Amidinophenyl)-5-[({[1-(1-(iminoethyl)-4-piperidyl}amino)methyl]benzoic acid dihydrochloride and process for preparing the same

(76) Inventors: Takayuki Hara, Tokyo (JP); Toru Minoshima, Yamaguchi (JP); Midori Kamimura, Tokyo (JP); Masayasu Tabe, Tokyo (JP); Yasunobu Takano, Tokyo (JP)

Correspondence Address: SUGHRUE MION, PLLC, 2100 PENNSYLVANIA AVENUE, N.W., SUITE 800, WASHINGTON, DC, 20037, US

(21) Appl. No.: 10/470,383

(22) PCT Filed: Jan. 28, 2002
(86) PCT No.: PCT/JP02/00606

(30) Foreign Application Priority Data
Jan. 30, 2001 (JP) 2001-021475

(51) Int. Cl. A61K 31/445; C07D 211/26

(52) U.S. Cl. 514/317; 546/229

(57) ABSTRACT

A process for preparing an amidine derivative represented by the following formula (II), wherein R represents a hydrogen atom or a phenyl group, which comprises reducing an amideoxime derivative represented by the following formula (I), wherein R represents a hydrogen atom or a phenyl group, with zinc in an acetic acid solvent, or a salt thereof.

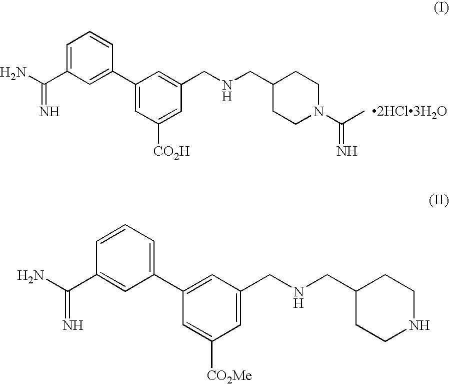

### FIELD OF THE INVENTION

The present invention relates to 3-(3-amidinophenyl)-5-[({[1-(1-iminoethyl)-4-piperidyl]methyl}amino)methyl]benzoic acid dihydrochloride. More particularly, it relates to 3-(3-amidinophenyl)-5-[({[1-(1-iminoethyl)-4-piperidyl]methyl}amino)methyl]benzoic acid dihydrochloride which is useful as an original drug of a novel selective inhibitor for activated coagulation factor X (hereinafter abbreviated to as “FXa”) and a process for preparing the same.

### BACKGROUND ART

Anti-thrombin agents have conventionally been developed as anti-thrombosis medicaments. However, it has been known that the anti-thrombin agents are likely to cause bleeding because they inhibit both an blood coagulation action and a platelet aggregation action by thrombin and thus they can not easily control a coagulation ability. Therefore, anticoagulants based on an action mechanism other than thrombin inhibition have been developed. Among them, biphenyl amidine derivatives described in the specification of International Publication Patent WO 99/26918 have been found as an anticoagulant having an excellent FXa inhibitory action.

By the way, if a single compound is generally in the form of solid, the single compound exists in crystal or amorphous states and a crystal may show crystal polymorphism. Since the stability and solubility of a compound vary depending on state, it is required to select one stable crystal form and to continually prepare it as an original drug for pharmaceutical preparations.

However, according to the process for preparing the biphenyl amidine derivatives described in the specification of International Publication Patent WO 99/26918, the final product is obtained by purifying using column chromatography and the resulting compound is an amorphous salt. In general, an amorphous salt is inferior in handling properties in a large amount because of its high hygroscopicity. Therefore, it has been required to develop a technique capable of giving one stable crystal form and effecting industrial mass production without purifying using column chromatography.

### DISCLOSURE OF THE INVENTION

An object of the present invention is to provide 3-(3-amidinophenyl)-5-[({[1-(1-iminoethyl)-4-piperidyl]methyl}amino)methyl]benzoic acid in one stable crystal form, which is a necessary condition in an original drug for pharmaceutical preparations, among the group of compounds having an physiological activity as a clinically applicable FXa inhibitor, described in the specification of International Publication Patent WO 99/26918, and a process for preparing the same. The present inventors have intensively studied processes which can provide a high-purity compound required in pharmaceutical preparations while satisfying the necessary condition described above and can also ensure mass production. As a result, the present inventors have found that this compound is crystallized if it is in a form of a dihydrochloride salt and also found a necessary condition which enables the dihydrochloride salt to give one stable crystal form. Thus, the present invention has been completed.

The present invention provides crystal of 3-(3-amidinophenyl)-5-[({[1-(1-iminoethyl)-4-piperidyl]methyl}amino)methyl]benzoic acid dihydrochloride hydrate which shows a main peak at a diffraction angle 2θ(°) of 12.2, 13.5, 16.5, 18.5, 19.2, 20.5, 22.0, 22.8, 23.6, 24.7, 25.1, 25.5, 26.1, 29.8, 33.1 and 33.7 in powder X-ray diffractometry, and a process for preparing the same.

The present invention also provides a process for preparing 3-(3-amidinophenyl)-5-[({[1-(1-iminoethyl)-4-piperidyl]methyl}amino)methyl]benzoic acid dihydrochloride hydrate, which comprises reacting methyl 3-(3-amidinophenyl)-5-({[(4-piperidyl)methyl]amino}methyl)benzoate represented by the following formula (II):

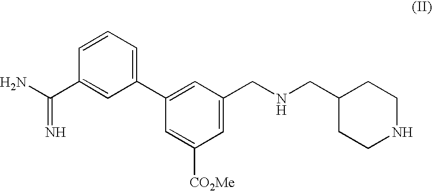

or a salt thereof with ethylacetoimidate hydrochloride to form methyl 3-(3-amidinophenyl)-5-[({[1-(1-iminoethyl)-4-piperidyl]methyl}amino)methyl]benzoate represented by the following formula (III):

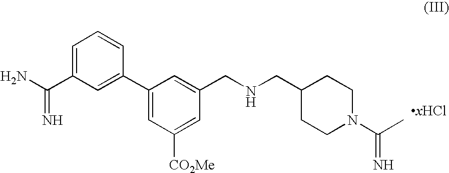

wherein x represents from 0 to 3, or a salt thereof, hydrolyzing the methyl ester with an acid, and subjecting the resulting hydrolysate to neutralization, purification by recrystallization, and moisture conditioning.

### BRIEF DESCRIPTION OF THE DRAWINGS

FIG. 1 is a graph showing a powder X-ray diffractometry spectrum of 3-(3-amidinophenyl)-5-[({[1-(1-iminoethyl)-4-piperidyl]methyl}amino)methyl]benzoic acid dihydrochloride.

FIG. 2 is a graph showing a powder X-ray diffractometry spectrum of 3-(3-amidinophenyl)-5-[({[1-(1-iminoethyl)-4-piperidyl]methyl}amino)methyl]benzoic acid dihydrochloride hydrate.

FIG. 3 is a graph showing a molecular structure of 3-(3-amidinophenyl)-5-[({[1-(1-iminoethyl)-4-piperidyl]methyl}amino)methyl]benzoic acid dihydrochloride trihydrate.

### MODE FOR CARRYING OUT THE INVENTION

A reaction for conversion of a compound of the above formula (II) into a compound of the above formula (III) is carried out by a process for reacting ethylacetoimidate hydrochloride in an alcohol solution in the presence of an amine. As the solvent, alcohol solvents such as methanol, ethanol and isopropyl alcohol can be used. Among these solvents, ethanol and methanol are preferred, and methanol is particularly preferred. As an amine which can be used in the reaction, tertiary amines such as trimethylamine, triethylamine, tributylamine and diisopropylethylamine can be used. Among these amines, triethylamine is preferred. The reaction can also be carried out with the co-existence of pyridine. If a compound represented by the above formula (II) is a complex with zinc chloride, a combination of triethylamine with pyridine is preferred. Reaction for hydrolysis of a compound represented by the above formula (III) is carried out in an acid solution. As the acid, hydrochloric acid, sulfuric acid and nitric acid can be used. Among these acids, hydrochloric acid is preferred.

A neutralization reaction after hydrolysis with an acid can be carried out using an alkali metal hydroxide such as sodium hydroxide, potassium hydroxide or lithium hydroxide, or an aqueous solution thereof, or a basic ion-exchange resin. Among these bases, an aqueous sodium hydroxide solution is preferred. Although the neutralization reaction can be carried out at a temperature within a range from 0 to 95° C., a desired product is solidified at low temperature and thus it becomes difficult to separate it from an insoluble matter. It becomes possible to separate only the insoluble matter by filtration when the neutralization operation is carried out in an aqueous solution at a temperature within a range from 35 to 60° C. and, therefore, the neutralization operation at a temperature within a range from 35 to 60° C. is preferred.

Furthermore, the pH of the aqueous solution is preferably maintained at a value within a range from 5.0 to 6.0 so as to obtain 3-(3-amidinophenyl)-5-[({[1-(1-iminoethyl)-4-piperidyl]methyl}amino)methyl]benzoic acid dihydrochloride in a good yield. In the purification and crystallization of 3-(3-amidinophenyl)-5-[({[1-(1-iminoethyl)-4-piperidyl]methyl}amino)methyl]benzoic acid dihydrochloride, an alcohol is added first to the solution after the neutralization operation to obtain 3-(3-amidinophenyl)-5-[({[1-(1-iminoethyl)-4-piperidyl]methyl}amino)methyl]benzoic acid dihydrochloride. The alcohol to be added is preferably ethanol or isopropyl alcohol. Among these alcohols, isopropyl alcohol is preferred.

Purification with recrystallization for improvement of the purity of 3-(3-amidinophenyl)-5-[({[1-(1-iminoethyl)-4-piperidyl]methyl}amino)methyl]benzoic acid dihydrochloride is carried out using an alcohol, water and acetic acid in combination. The alcohol used for crystallization is preferably ethanol or isopropyl alcohol. Among these alcohols, ethanol is preferred. The recrystallization makes it possible to purify without using column chromatography. A crystal obtained by using acetic acid and an alcohol in combination has higher solubility than that of a crystal obtained by using water.

Therefore, an aqueous solution can be prepared from the crystal in a smaller amount than that of the crystal obtained from water at lower temperature where decomposition scarcely occurs. In this case, acetic acid used in the recrystallization may contain water in an amount of 40% or less, and preferably 30% or less.

The final recrystallization is carried out by using a reprecipitation process by the addition of a poor solvent or a vapor replacement process after dissolving 3-(3-amidinophenyl)-5-[({[1-(1-iminoethyl)-4-piperidyl]methyl}amino)methyl]benzoic acid dihydrochloride in water. The resulting crystal is subjected to both of the removal of an organic solvent under reduced pressure and moisture control, thus making it possible to obtain 3-(3-amidinophenyl)-5-[({[1-(1-iminoethyl)-4-piperidyl]methyl}amino)methyl]benzoic acid dihydrochloride hydrate in a stable crystal form.

In this case, 3-(3-amidinophenyl)-5-[({[1-(1-iminoethyl)-4-piperidyl]methyl}amino)methyl]benzoic acid dihydrochloride hydrate contains water in a predetermined amount in terms of 0.5 to 3.5 hydrate, and preferably 2.5 to 3.5 hydrate. Examples of the poor solvent used in the recrystallization include methanol, ethanol, isopropyl alcohol, N,N-dimethylformamide, N-methylpylloridine and acetone. Among these poor solvents, ethanol and isopropyl alcohol are preferred and ethanol is particularly preferred.

The crystal of 3-(3-amidinophenyl)-5-[({[1-(1-iminoethyl)-4-piperidyl]methyl}amino)methyl]benzoic acid dihydrochloride is useful as an original drug for pharmaceutical preparations because it is stable in atmospheric air and can endure storage for a long period. Furthermore, 3-(3-amidinophenyl)-5-[({[1-(1-iminoethyl)-4-piperidyl]methyl}amino)methyl]benzoic acid dihydrochloride of the present invention includes various pharmaceutically acceptable solvates, and those which may show crystal polymorphism.

The present invention also provides a crystal of 3-(3-amidinophenyl)-5-[({[1-(1-iminoethyl)-4-piperidyl]methyl}amino)methyl]benzoic acid dihydrochloride which shows a main peak at a diffraction angle 2θ(°) of 16.2, 17.1, 18.3, 19.0, 20.5, 21.1, 22.7, 23.2, 24.7, 25.6, 28.4, 29.5, 33.2, 34.3 and 35.8 in powder X-ray diffractometry, the crystal being obtained by a recrystallization process comprising adding an alcohol after dissolving 3-(3-amidinophenyl)-5-[({[1-(1-iminoethyl)-4-piperidyl]methyl}amino)methyl]benzoic acid dihydrochloride in acetic acid which may contain not more than 30% of water among the processes described above.

The present invention also provides crystal of 3-(3-amidinophenyl)-5-[({[1-(1-iminoethyl)-4-piperidyl]methyl}amino)methyl]benzoic acid dihydrochloride hydrate which shows a main peak at a diffraction angle 2θ(°) of 12.2, 13.5, 16.5, 18.5, 19.2, 20.5, 22.0, 22.8, 23.6, 24.7, 25.1, 25.5, 26.1, 29.8, 33.1 and 33.7 in powder X-ray diffractometry, and is useful as an original drug for pharmaceutical preparations, the crystal being obtained by the process described above.

The present invention also provides a crystal of 3-(3-amidinophenyl)-5-[({[1-(1-iminoethyl)-4-piperidyl]methyl}amino)methyl]benzoic acid dihydrochloride trihydrate, wherein the 3-(3-amidinophenyl)-5-[({[1-(1-iminoethyl)-4-piperidyl]methyl}amino)methyl]benzoic acid dihydrochloride hydrate described above is represented by the following formula (I):

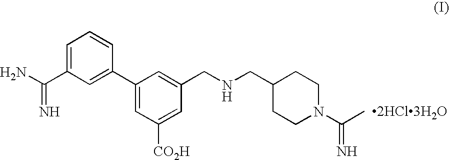

### EXAMPLES

The present invention will be described in detail by way of the following examples. However, the present invention is not limited thereto.

### EXAMPLE 1

### 3-(3-amidinophenyl)-5-[({[1-(1-iminoethyl)-4-piperidyl]methyl}amino)methyl]benzoic Acid Dihydrochloride (Compound of claim 2)

97.93 g of methyl 3-(3-amidinophenyl)-5-[({[1-(1-iminoethyl)-4-piperidyl]methyl}amino)methyl]benzoate 1.5 zinc chloride trihydrochloride dihydrate (obtained from the compound described in the specification of International Publication Patent WO 99/26918 by a known procedure) was dissolved in 1 L of methanol and 72.5 mL of pyridine, 70.5 g of ethylacetoimidate hydrochloride and 203 mL of triethylamine were added, followed by stirring at room temperature for 5 hours. The reaction mixture was directly concentrated under reduced pressure. 500 mL of concentrated (35 to 375) hydrochloric acid was added to the concentrated mixture, followed by stirring at room temperature for 14 hours.

Subsequently, the reaction mixture was heated to 95° C. and stirred for 8 hours. The reaction mixture was directly concentrated under reduced pressure. 550 mL of water was added to the concentrated mixture and then dissolved. While heating the mixture to 40 to 45° C., 142 mL of 4 mol/L of an aqueous sodium hydroxide solution was added to adjust the pH of the mixture to a value within a range from 5.4 to 5.6. The insoluble matter deposited as a result of neutralization was removed by filtration.

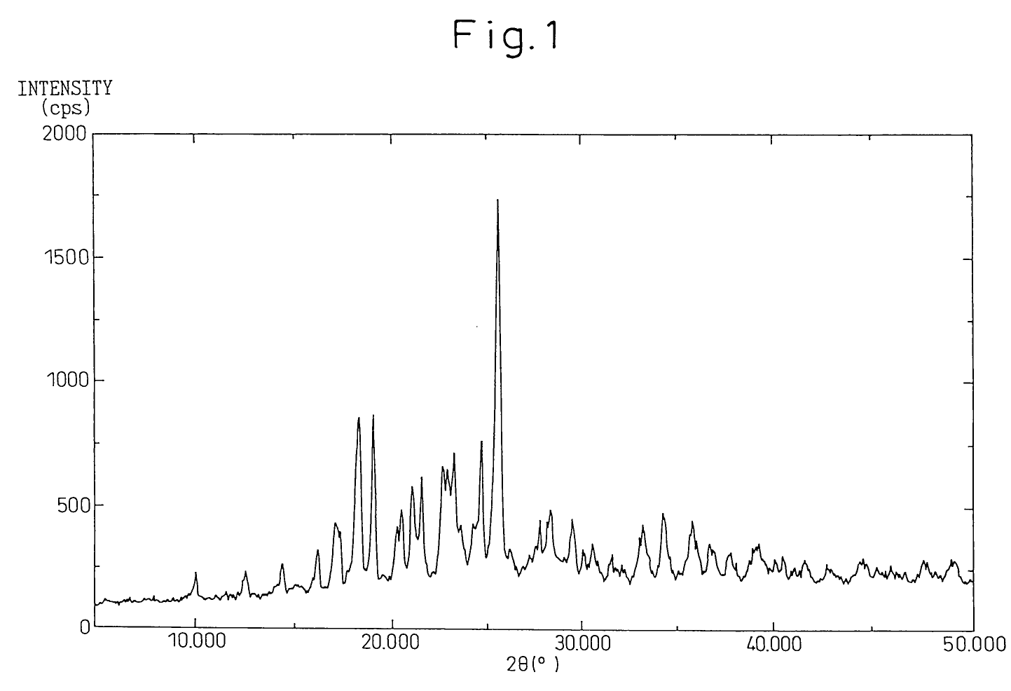

After heating the filtrate to 80° C., 1.48 L of isopropyl alcohol was added. The solution was slowly cooled to room temperature while stirring, and then stirred at room temperature for an additional 14 hours. The crystal deposited by addition of isopropyl alcohol was dissolved in 310 mL of acetic acid at 80° C. 1.2 L of ethanol was added to the solution, followed by stirring at room temperature for 14 hours. The crystal deposited by the addition of ethanol was dried at 50° C. under reduced pressure to obtain 141.5 g of the title compound. The resulting powder X-ray diffractometry spectrum is shown in FIG. 1, and IR and NMR analysis data are shown below.

IR (KBr, cm⁻¹) of 1680, 1626, 1570, 1383. 1H-NMR (600 MHz, δppm, CD3OD/TMS) of 1.39-1.48 (m, 2H), 2.01-2.05 (m, 2H), 2.18-2.21 (m, 1H), 2.32 (s, 3H), 3.01 (d, J=7.2 Hz, 2H), 3.17-3.22 (m, 1H), 3.28-3.34 (m, 1H), 4.05-4.10 (m, 2H), 4.29 (s, 2H), 7.74 (dd, J=8.4 & 7.2 Hz, 1H), 7.82 (d, J=7.8 Hz, 1H), 8.03 (s, 1H), 8.07 (s, 1H), 8.11 (dt, J=8.4 & 1.2 Hz, 1H), 8.17 (t, J=1.2 Hz, 1H), 8.33 (s, 1H).

### EXAMPLE 2

### 3-(3-amidinophenyl)-5-[({[1-(1-iminoethyl)-4-piperidyl]methyl}amino)methyl]benzoic Acid Dihydrochloride Hydrate (Compound of claim 1)

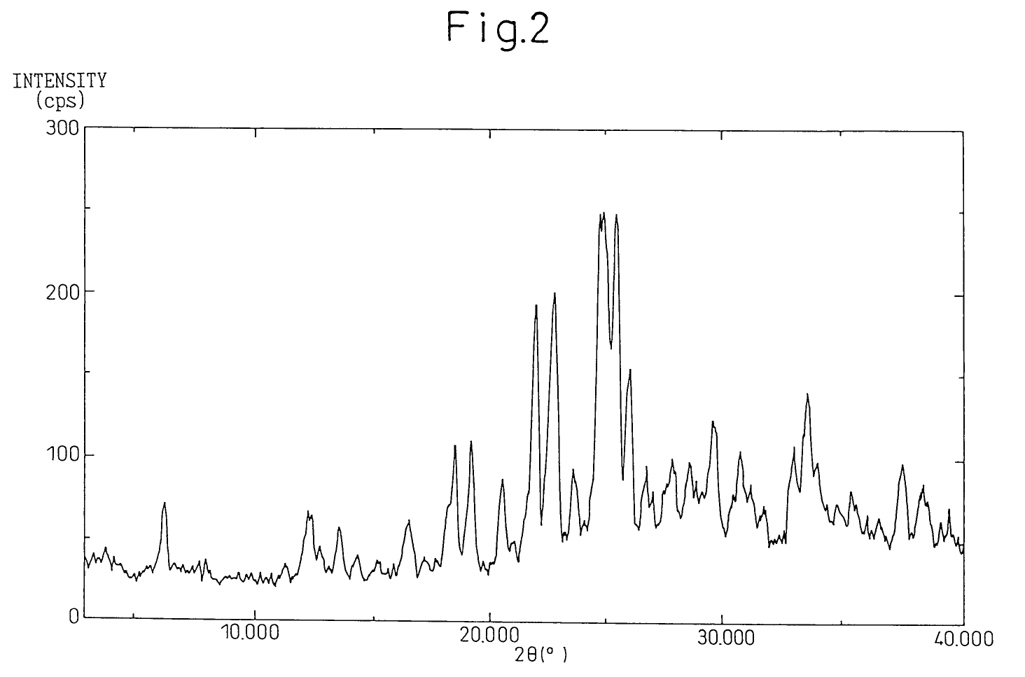

3.06 g of 3-(3-amidinophenyl)-5-[({[1-(1-iminoethyl)-4-piperidyl]methyl}amino)methyl]benzoic acid dihydrochloride was dissolved in 12 mL of water added by heating to 50° C. The insoluble matter was removed by filtration while being kept in a hot condition and 48 mL of ethanol was added, followed by stirring for 4 hours while maintaining at a temperature of 50° C. The crystal deposited was collected by filtration, dried under reduced pressure and allowed to stand until the weight of the crystal becomes constant in a bath maintained at constant humidity of 75% to obtain 2.74 g of the title compound. The resulting powder X-ray diffractometry spectrum is shown in FIG. 2, and IR, NMR and elemental analysis data are shown below.

IR (KBr, cm⁻¹) of 1705, 1570, 1391, 700. 1H-NMR (600 MHz, δppm, CD3OD/TMS) of 1.39-1.48 (m, 2H), 2.01-2.05 (m, 2H), 2.18-2.21 (m, 1H), 2.32 (s, 3H), 3.01 (d, J=7.2 Hz, 2H), 3.17-3.22 (m, 1H), 3.28-3.34 (m, 1H), 4.05-4.10 (m, 2H), 4.29 (s, 2H), 7.74 (dd, J=8.4 & 7.2 Hz, 1H), 7.82 (d, J=7.8 Hz, 1H), 8.03 (s, 1H), 8.07 (s, 1H), 8.11 (dt, J=8.4 & 1.2 Hz, 1H), 8.17 (t, J=1.2 Hz, 1H), 8.33 (s, 1H).

Elemental analysis of (C₂₃H₂₉N₅O₂2HCl2.8H₂O); Calculated of C (52.04), H (6.95), N (13.19), Cl (13.36) Found of C (51.97), H (6.91), N (12.68), Cl (13.25).

### EXAMPLE 3

### 3-(3-amidinophenyl)-5-[({[1-(1-iminoethyl)-4-piperidyl]methyl}amino)methyl]benzoic Acid Dihydrochloride Trihydrate

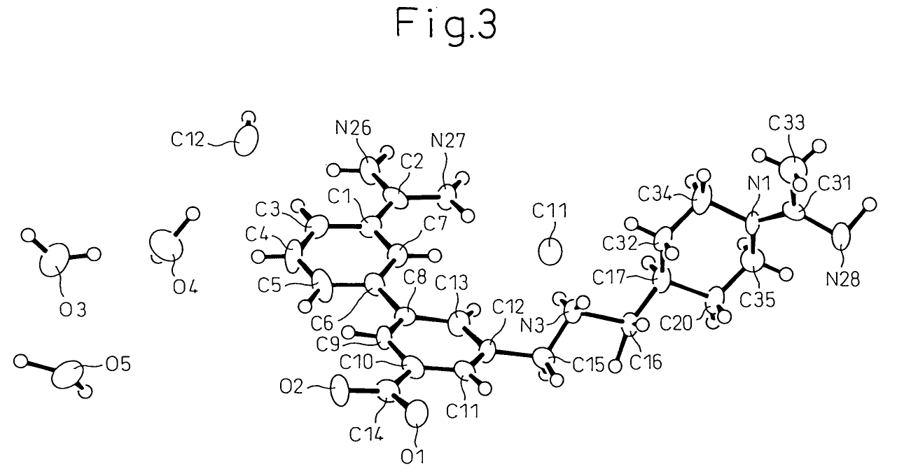

0.99 g of 3-(3-amidinophenyl)-5-[({[1-(1-iminoethyl)-4-piperidyl]methyl}amino)methyl]benzoic acid dihydrochloride, was dissolved in 10 mL of water added by heating to 90° C. 35 mL of ethanol was added while maintaining the same temperature. After the stirring was stopped, the mixed solution was slowly cooled to room temperature and allowed to stand for 2 days, thereby to cause crystallization. After the solvent was removed, the residue was dried under reduced pressure and moisture control was carried out by being left to stand until the weight of the crystal becomes constant in a bath maintained at constant humidity of 75% to obtain 0.77 g of the title compound. The crystal size was 0.10×0.05×0.20 mm³. The molecular structure is shown in FIG. 3, and 1H-NMR analysis data and X-ray crystal analysis data are shown bellow.

1H-NMR was determined by dissolving 1.00 mg of 3-(3-amidinophenyl)-5-[({[1-(1-iminoethyl)-4-piperidyl]methyl}amino)methyl]benzoic acid dihydrochloride trihydrate in 0.5 mL of methanol-d4.

1H-NMR (600 MHz, δppm, CD3OD/TMS) of 1.39-1.48 (m, 2H), 2.01-2.05 (m, 2H), 2.18-2.21 (m, 1H), 2.32 (s, 3H), 3.01 (d, J=7.2 Hz, 2H), 3.17-3.22 (m, 1H), 3.28-3.34 (m, 1H), 4.05-4.10 (m, 2H), 4.29 (s, 2H), 7.74 (dd, J=87.4 & 7.2 Hz, 1H), 7.82 (d, J=7.8 Hz, 1H), 8.03 (s, 1H), 8.07 (s, 1H), 8.11 (dt, J=8.4 & 1.2 Hz, 1H), 8.17 (t, J=1.2 Hz, 1H), 8.33 (s, 1H).

Crystallographic Data:

Space froup C2/c

z=8

a=30.677(6) Å

b=7.234(5) Å

c=24.962(5) Å

β=109.22(1) °

ν=5230(4) Å3

Total Reflections: 4338

Unique 3885

Rl=0.074

X-ray crystal analysis data

TABLE 1
Atomic coordinates, Bᵢ₉₀/B_{eq} and occupancy

| atom | x | y | z | B_{eq} | occ |
|---|---|---|---|---|---|
| C1(1) | 0.88398(8) | −0.0076(4) | 0.00836(9) | 3.84(5) | 1.0000 |
| C1(2) | 0.59150(7) | 0.1548(4) | −0.20634(9) | 3.90(6) | 1.0000 |
| O(1) | 0.8079(2) | −0.0799(8) | 0.3040(2) | 2.8(1) | 1.0000 |
| O(2) | 0.7339(2) | −0.0588(9) | 0.2494(2) | 3.7(2) | 1.0000 |
| O(3) | 0.4597(2) | 0.168(1) | 0.0282(3) | 6.5(2) | 1.0000 |
| O(4) | 0.5540(2) | 0.1885(10) | 0.0260(3) | 5.5(2) | 1.0000 |
| O(5) | 0.5000 | 0.111(1) | 0.2500 | 6.7(3) | 0.5000 |
| N(1) | 1.0788(2) | 0.281(1) | 0.1324(3) | 2.9(2) | 1.0000 |
| N(3) | 0.9216(2) | −0.0125(10) | 0.1428(2) | 2.5(2) | 1.0000 |
| N(26) | 0.7055(2) | 0.079(1) | −0.1566(3) | 3.3(2) | 1.0000 |
| N(27) | 0.7791(2) | 0.051(1) | −0.1008(3) | 3.4(2) | 1.0000 |
| N(28) | 1.1570(2) | 0.279(1) | 0.1766(3) | 3.6(2) | 1.0000 |
| C(1) | 0.7191(3) | 0.039(1) | −0.0566(3) | 2.2(2) | 1.0000 |
| C(2) | 0.7354(3) | 0.057(1) | −0.1058(3) | 2.6(2) | 1.0000 |
| C(3) | 0.6727(3) | 0.041(1) | −0.0640(3) | 3.6(2) | 1.0000 |
| C(4) | 0.6577(3) | 0.014(2) | −0.0181(4) | 4.3(2) | 1.0000 |
| C(5) | 0.6893(3) | −0.018(2) | 0.0354(3) | 3.9(2) | 1.0000 |
| C(6) | 0.7366(2) | −0.021(1) | 0.0443(3) | 2.4(2) | 1.0000 |
| C(7) | 0.7506(3) | 0.007(1) | −0.0026(3) | 2.7(2) | 1.0000 |
| C(8) | 0.7706(3) | −0.049(1) | 0.1016(3) | 2.3(2) | 1.0000 |
| C(9) | 0.7589(3) | −0.040(1) | 0.1506(3) | 2.5(2) | 1.0000 |
| C(10) | 0.7911(3) | −0.072(1) | 0.2042(3) | 2.5(2) | 1.0000 |
| C(11) | 0.8360(3) | −0.100(1) | 0.2082(3) | 2.6(2) | 1.0000 |
| C(12) | 0.8499(2) | −0.110(1) | 0.1613(3) | 2.2(2) | 1.0000 |
| C(13) | 0.8166(3) | −0.088(1) | 0.1078(3) | 2.7(2) | 1.0000 |

TABLE 2
Continuing from Table 1

| atom | x | y | z | B_{eq} | occ |
|---|---|---|---|---|---|
| C(14) | 0.7759(3) | −0.070(1) | 0.2562(3) | 2.7(2) | 1.0000 |
| C(15) | 0.8988(3) | −0.156(1) | 0.1699(3) | 2.6(2) | 1.0000 |
| C(16) | 0.9720(3) | −0.054(1) | 0.1566(4) | 3.5(2) | 1.0000 |
| C(17) | 0.9976(3) | 0.060(1) | 0.1269(3) | 2.8(2) | 1.0000 |
| C(20) | 1.0455(3) | −0.021(1) | 0.1404(4) | 2.9(2) | 1.0000 |
| C(31) | 1.1171(3) | 0.361(1) | 0.1604(4) | 2.9(2) | 1.0000 |
| C(32) | 1.0016(3) | 0.267(1) | 0.1429(4) | 3.5(2) | 1.0000 |
| C(33) | 1.1171(3) | 0.563(1) | 0.1721(4) | 4.0(2) | 1.0000 |
| C(34) | 1.0326(3) | 0.368(1) | 0.1175(4) | 3.9(2) | 1.0000 |
| C(35) | 1.0753(3) | 0.088(1) | 0.1144(4) | 3.4(2) | 1.0000 |
| H(1) | 0.5464 | 0.1010 | −0.0062 | 0.0000 | 1.0000 |
| H(2) | 0.9086 | 0.0035 | 0.1028 | 0.0000 | 1.0000 |
| H(3) | 0.6256 | −0.0070 | −0.0257 | 0.0000 | 1.0000 |
| H(5) | 0.9778 | 0.0435 | 0.0867 | 0.0000 | 1.0000 |
| H(7) | 0.6506 | 0.0622 | −0.1010 | 0.0000 | 1.0000 |
| H(8) | 0.6790 | −0.0468 | 0.0065 | 0.0000 | 1.0000 |
| H(9) | 0.7825 | 0.0010 | 0.0009 | 0.0000 | 1.0000 |
| H(10) | 0.7290 | −0.0123 | 0.1479 | 0.0000 | 1.0000 |
| H(11) | 0.8580 | −0.1151 | 0.2453 | 0.0000 | 1.0000 |
| H(12) | 0.8274 | −0.1029 | 0.0762 | 0.0000 | 1.0000 |
| H(13) | 0.6742 | 0.0952 | −0.1619 | 0.0000 | 1.0000 |
| H(14) | 0.7169 | 0.0867 | −0.1881 | 0.0000 | 1.0000 |
| H(15) | 0.8011 | 0.0408 | −0.0630 | 0.0000 | 1.0000 |
| H(16) | 0.7896 | 0.0569 | −0.1320 | 0.0000 | 1.0000 |
| H(17) | 1.0370 | 0.4918 | 0.1324 | 0.0000 | 1.0000 |

TABLE 3
Continuing from Table 2

| atom | x | y | z | B_{eq} | occ |
|---|---|---|---|---|---|
| H(18) | 1.0192 | 0.3728 | 0.0778 | 0.000 | 1.0000 |
| H(19) | 0.8994 | −0.2725 | 0.1483 | 0.000 | 1.0000 |
| H(20) | 0.9168 | −0.1716 | 0.2063 | 0.000 | 1.0000 |
| H(21) | 0.9862 | −0.0426 | 0.1965 | 0.000 | 1.0000 |
| H(22) | 0.9747 | −0.1843 | 0.1472 | 0.000 | 1.0000 |
| H(23) | 1.0602 | −0.0192 | 0.1808 | 0.000 | 1.0000 |
| H(24) | 1.0432 | −0.1454 | 0.1277 | 0.000 | 1.0000 |
| H(25) | 1.0615 | 0.0835 | 0.0732 | 0.000 | 1.0000 |
| H(26) | 1.1053 | 0.0358 | 0.1239 | 0.000 | 1.0000 |
| H(27) | 0.9710 | 0.3197 | 0.1297 | 0.000 | 1.0000 |
| H(28) | 1.0128 | 0.2729 | 0.1831 | 0.000 | 1.0000 |
| H(31) | 0.9257 | 0.0927 | 0.1695 | 0.000 | 1.0000 |
| H(35) | 1.1295 | 0.5806 | 0.2118 | 0.000 | 1.0000 |
| H(36) | 1.1373 | 0.6227 | 0.1548 | 0.000 | 1.0000 |
| H(37) | 1.0875 | 0.6125 | 0.1574 | 0.000 | 1.0000 |
| H(38) | 0.4361 | 0.2715 | 0.0214 | 0.000 | 1.0000 |
| H(39) | 0.4921 | 0.1783 | 0.0267 | 0.000 | 1.0000 |
| H(40) | 0.4718 | 0.0219 | 0.2367 | 0.000 | 1.0000 |
| H(41) | 0.5282 | 0.0219 | 0.2633 | 0.000 | 1.0000 |
| H(42) | 0.5750 | 0.2910 | 0.0130 | 0.000 | 1.0000 |
| H(44) | 1.1869 | 0.2214 | 0.1892 | 0.000 | 1.0000 |
| H(45) | 0.5882 | 0.1720 | −0.2472 | 0.000 | 1.0000 |

B_{eq}=(8/3)π²(U₁₁(aa*)²+U₂₂(bb*)²+U₃₃(cc*)²+2U₁₂aa*bb*cosγ+2U₁₃aa*cc*cosβ+2U₂₃bb*cc*cosα)

### EXAMPLE 4

### 3-(3-amidinophenyl)-5-[({[1-(1-iminoethyl)-4-piperidyl]methyl}amino)methyl]benzoic Acid Dihydrochloride Trihydrate

7.77 mg of 3-(3-amidinophenyl)-5-[({[1-(1-iminoethyl)-4-piperidyl]methyl}amino)methyl]benzoic acid dihydrochloride trihydrate was charged in a small vessel (sample bottle) and dissolved in 0.5 mL of water. 7 mL of acetone was charged in a wide-necked conical flask and the sample bottle was arranged in the conical flask so that a portion of the outside of the sample bottle is immersed (the aqueous solution is not directly mixed with acetone).

The opening of the conical flask was covered with Parafilm to seal the conical flask and the flask was allowed to stand for 4 weeks. A portion of acetone evaporated in the conical flask was absorbed in the aqueous solution and a crystal having a size required for analysis of the crystal could be obtained. The size of the crystal used in the analysis was 0.1×0.1×0.1 mm³.

Crystallographic Data are as Follows:

Space froup C2/c

z=8

a=30.63(1) Å

b=7.231(9) Å

c=24.95(1) Å

β=109.23(3) °

ν=5218(7) Å3

Total Reflections: 4340

Unique 3884

Rl=0.074

The crystal analysis data and NMR data show that the resulting crystal has the same structure as in Example 3.

### INDUSTRIAL APPLICABILITY

According to the present invention, it is possible to prepare a large amount of 3-(3-amidinophenyl)-5-[({[1-(1-iminoethyl)-4-piperidyl]methyl}amino)methyl]benzoic acid, which is a compound having a physiological activity as a clinically applicable FXa inhibitor, in a high-quality and stable crystal form without purifying with column chromatography. Therefore, the present invention is industrially useful.

1. Crystal of 3-(3-amidinophenyl)-5-[({[1-(1-iminoethyl)-4-piperidyl]methyl}amino)methyl]benzoic acid dihydrochloride hydrate, which shows main peaks at a diffraction angle 2θ(°) of 12.2, 13.5, 16.5, 18.5, 19.2, 20.5, 22.0, 22.8, 23.6, 24.7, 25.1, 25.5, 26.1, 29.8, 33.1 and 33.7 in powder x-ray diffractometry.

2. Crystal of 3-(3-amidinophenyl)-5-[({[1-(1-iminoethyl)-4-piperidyl]methyl}amino)methyl]benzoic acid dihydrochloride, which shows main peaks at a diffraction angle 2θ(°) of 16.2, 17.1, 18.3, 19.0, 20.5, 21.1, 22.7, 23.2, 24.7, 25.6, 28.4, 29.5, 33.2, 34.3 and 35.8 in powder X-ray diffractometry.

3. Crystal of 3-(3-amidinophenyl)-5-[({[1-(1-iminoethyl)-4-piperidyl]methyl}amino)methyl]benzoic acid dihydrochloride trihydrate, according to claim 1 represented by the following formula (I):

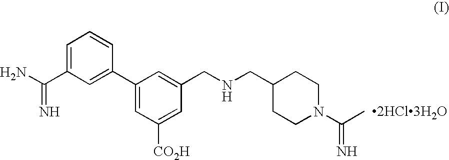

4. A process for preparing 3-(3-amidinophenyl)-5-[({[1-(1-iminoethyl)-4-piperidyl]methyl}amino)methyl]benzoic acid dihydrochloride hydrate of claim 1, which comprises reacting methyl 3-(3-amidinophenyl)-5-({[(4-piperidyl)methyl]amino}methyl)benzoate represented by the following formula (II): 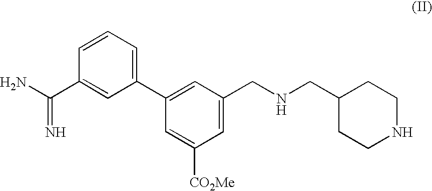
or a salt thereof with ethylacetoimidate hydrochloride to form methyl 3-(3-amidinophenyl)-5-[({[1-(1-iminoethyl)-4-piperidyl]methyl}amino)methyl]benzoate represented by the following formula (III): 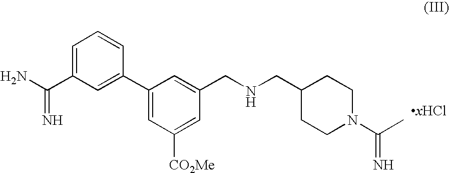
wherein x represents 0 to 3, or a salt thereof, hydrolyzing the methyl ester with an acid, and subjecting the resulting hydrolysate to neutralization, purification by recrystallization and moisture conditioning.

5. A process for preparing the crystal of claim 1, which comprises dissolving 3-(3-amidinophenyl)-5-[({[1-(1-iminoethyl)-4-piperidyl]methyl}amino)methyl]benzoic acid dihydrochloride in water to form a solution, and adding an alcohol to the solution.

6. A process for preparing the crystal of claim 2, which comprises dissolving 3-(3-amidinophenyl)-5-[({[1-(1-iminoethyl)-4-piperidyl]methyl}amino)methyl]benzoic acid dihydrochloride in acetic acid which may contain 30% or less of water to form a solution, and adding an alcohol to the solution.
